(19) United States
(12) Patent Application Publication
(10) Pub. No.: US 2009/0216069 A1
(43) Pub. Date: Aug. 27, 2009

(54) ENCLOSURE FOR SURGICAL PROCEDURES

(76) Inventors: John Patrick Woodcock, Wales (GB); Brian James Western, Wales (GB); Anurag Srivastava, Ansari Nagar (IN)

Correspondence Address: KING & SCHICKLI, PLLC, 247 NORTH BROADWAY, LEXINGTON, KY, 40507, US

(21) Appl. No.: 12/161,920

(22) PCT Filed: Jan. 8, 2007
(86) PCT No.: PCT/GB2007/000039

§ 371 Date: Nov. 7, 2008

(30) Foreign Application Priority Data
Jan. 25, 2006 (GB) 0601479.9
Jul. 6, 2006 (GB) 0613460.5

(51) Int. Cl. A61B 19/00

(52) U.S. Cl. 600/21

(57) ABSTRACT

The invention relates to a medical device for performing substantially bloodless surgery wherein the device comprises a double-skinned chamber with at least one through-bore.

The invention relates to a medical device for performing substantially bloodless surgery.

Surgeons endeavour to minimise operative blood loss by making any incision neat and precise. However, under normal circumstances blood loss is unavoidable and the amount of blood loss governs the degree of occlusion that takes place and thus the amount of inadvertent injury to any structure, vital or otherwise, obscured in a pool of blood. Despite these best endeavours, it is known for injury to the recurrent laryngeal nerve to occur during thyroidectomy, for injury to the bile duct during cholycystectomy and injury to the ureter during pelvic surgery. Various techniques are employed to reduce blood loss such as, as mentioned, scrupulous dissection which typically involves controlling the blood vessels before cutting, but other techniques are also used such as electro-cautery, argon beam coagulation, ultrasonic dissection, the use of lasers and haemostatic agents such as cellulose, gelatin and collagen. However, these techniques only minimise blood loss and are sometimes not effective as in the case of large vascular tumours, haemangioma and arteriovenous malformations.

The unavoidable loss of a significant amount of blood volume necessitates replacement with blood from a compatible tissue type. But even where replacement is possible, massive transfusion can be associated with complications like coagulation derangement, hyperkalaemia and hypothermia. Moreover, compatible transfusion also carries the risk of transmission of microbial infections such as HIV, Hepatitis and Malaria.

It follows from the above that if the surgeon was able to perform bloodless, or substantially bloodless, surgery then one could avoid the operative field being occluded by pools of blood and so aid the surgeon by reducing the possibility of inadvertent injury. Additionally, one would also reduce the operative time and further, one could avoid the loss of significant amounts of blood and therefore the need for transfusion.

Bloodless surgery is therefore a highly desirable goal and brings with it any one or more of the following advantages.

It greatly reduces the need for the collection and storage of blood; it would overcome some of the difficulties associated with finding an adequate supply of blood that was classified as being from a rare blood group; it would make surgery a more feasible option for those with religious beliefs that prevent them from having blood transfusions; it would reduce the trauma and complications associated with blood loss and blood transfusion; it would safeguard against the contamination of staff with infections contracted by contact with contaminated blood; it would provide a clean environment in which to perform surgery; it would make surgery possible at the site of injury in impoverished countries where facilities are poor, and it would redistribute blood away from the operative site and thereby help maintain central blood pressure.

Despite these highly desirable advantages, hitherto, surgery has always been performed in such a way that an inevitable by product is the loss of blood and the associated complications.

Regardless of this status quo we have developed a medical device that enables surgery to be performed in a bloodless, or substantially bloodless, environment. By this we mean that we are able to perform surgery without the loss of blood or without a significant amount of bleeding such that the operative field remains largely unobscured and the surgeon is therefore able to see tissue to be treated or operated upon.

Our medical device is suitable for use both in the operating theatre and in the field. In the former instance our medical device can be adapted for use by either a surgeon or a robot. This is also true in the field but, typically, in the extreme conditions where field surgery has to be performed it will normally be performed by a surgeon. For example, where an individual has suffered an accident in a remote location and surgery needs to be performed immediately, then our medical device will enable a field surgeon to perform an operation in a bloodless or substantially bloodless environment.

Additionally, our medical device is used in stabilising a wound prior to treatment. For example, where an individual that has suffered an accident such as a car accident, or the like, and sustained a wound then the medical device can be placed about the wound in order to prevent any further blood loss and also promote healing.

We have found that our medical device actually promotes healing due to high pressure oxygen and therefore it is used to advantage during surgery. For example, it may be used during abdominal operations especially those associated with significant bleeding viz. resection of tumour of liver, adrenal, renal or retroperitoneum.

### STATEMENTS OF INVENTION

According to a first aspect of the invention there is provided a medical device for performing substantially bloodless surgery comprising an inflatable chamber having a through-bore adapted to receive a body part of an individual to be operated upon, which chamber, in use, is in fluid communication with a pressurised gas supply for inflating same and maintaining pressure in said chamber at a selected level wherein, upon inflation of said chamber the inner and outer walls thereof are forced thereapart and the inner wall sealingly engages with the site to be operated upon and the outer wall defines at least a part of a pressurised chamber in which surgery can take place; and further wherein said chamber includes at least one portal that allows access to the inside of the chamber, without significantly affecting the maintenance of said pressure, whereby said surgery can take place.

In a preferred embodiment of the invention the inner wall is made of a material that can be cut with a scalpel whereas the outer wall comprises a material that upon reaching a certain size will resist further deformation.

According to a second aspect of the invention there is provided a medical device for performing substantially bloodless surgery comprising an inflatable double-skinned chamber which, in use, is in fluid communication, via a single inlet means, with a pressurised gas supply for inflating same and maintaining pressure in said chamber at a selected level; said chamber being adapted to be placed sealingly against or about an individual to be treated and including at least one portal that allows access to the inside of said chamber, without significantly affecting the maintenance of said pressure, whereby said individual can be treated characterised in that;

said chamber comprises an inner and an outer skin, the inner skin being made of a relatively thin material that can be cut with a scalpel and the outer skin being made of a material that resists deformation on inflation whereby on inflation of said chamber said inner and outer skins are forced thereapart whereby said inner skin sealingly engages with the site to be operated upon and the outer skin defines a pressurised cavity in which surgery takes place.

Typically, said inner and outer walls or skins comprise two separate materials. However, it is within the scope of the invention for the inner and outer walls or skins to be made of the same material providing the material fulfils all of the stated properties. Thus one can use a single material that is suitably thin to be cut with a scalpel but also resistant to deformation on inflation. However, more usually, two separate materials will be used and the inner material is most usually a plastics material of a transparent and thin nature. As an alternative a thin latex material could be used but it would, preferably, have to be of a sort that does not elicit an allergic reaction. The outer wall or skin is most usually a plastics material although, because this material does not have direct contact with a patient's skin it can be a latex material providing, in either case, that it resists extension at the pressures that are used in order to perform the surgery. These pressures are typically no greater than systolic pressure, including systolic pressure of an individual experiencing high blood pressure but, most typically, especially where superficial surgery takes place to remove moles, epithelial growths, correct varicose veins or remove other surface lesions such as cancers and the like, then pressures significantly below systolic may be used because the surgeon is unlikely to be cutting an artery or a vein, rather the surgeon will be cutting through smaller blood vessels such as capillaries.

In use the medical device is placed sealingly against or about a part of an individual using the inner wall or skin as a seal. Whilst it is desirable that the seal is as tight as possible, it need not be hermetic provided the pressure within the chamber can be maintained at a selected level. In other words, some leakage of gas can be tolerated provided it can be replaced by the pressurised gas supply in order to maintain the elevated pressure within the chamber. Further, techniques are known for maintaining a seal about or against the body of an individual and they may include the use of creams, emulsions, suspensions or the like which when placed on the body of an individual help to form a tighter seal with a sealing member.

In use, once the medical device has been inflated the surgeon uses said portal in order to gain access to the pressurised chamber and, using a scalpel, cuts through both the inner wall or skin and the patient's tissue in order to gain access to the organ or tissue to be treated. It is therefore desirable for the inner wall or skin to be made of a thin material that can be cut with a scalpel but also a material with relatively clinging properties so that upon exposure to a pressurised gas the material is pushed sealingly against the skin of a patient and, in effect, acts as a second skin. This means that once incision has taken place the patient's tissues are exposed to the pressure inside the medical device.

It will therefore be apparent to one skilled in the art that the medical device of the invention acts to reverse the pressure gradient across vascular tissue and so prevent the loss of blood from the vascular system by maintaining an elevated pressure, which is greater than the tissue blood pressure at the site of the operation or, ideally, an elevated pressure to a maximum that approximates to, or about, systolic blood pressure; so ensuring that leakage of blood is avoided and therefore the area within the chamber can be treated without the operative field being obscured.

In a preferred embodiment of the invention said medical device is a transparent chamber.

Reference herein to a transparent chamber includes reference to a chamber that is transparent to the extent that a surgeon can see into the chamber sufficiently to perform surgery. It is therefore not necessary for the entire chamber to be transparent and, indeed, conventionally, modern surgery is typically undertaken by restricting the operative field to an area of tissue that is delineated by the use of surgical drapes. It is therefore sufficient for the chamber to be transparent to the extent that the surgeon is simply able to perform the operation. Although, in some embodiments of the invention the entire chamber is transparent; this is particularly preferred where more than one individual has access to the inside of said chamber.

In an alternative aspect of the invention the chamber may be opaque or lacking any transparency. This particular version of the invention is suited for use in relation to robotic surgery and, ideally, associated camera means are provided whereby a surgeon can operate the robot without needing to see through the walls of the medical device.

In a preferred embodiment of the invention said medical device is provided with a pressure monitoring means in order to monitor the maintenance of said pressure at said selected level and, ideally, venting means for venting pressurised gas within said chamber. As will be apparent to those skilled in the art the selective control of the supply of pressured gas and the venting thereof enables the maintenance of pressure inside said chamber at said selected level. Moreover, in an alternative embodiment of the invention a closed circuit gas system may be provided which reduces the amount of gas required to run the system.

Advantageously, we have discovered that whilst our medical device, through manipulation of the external pressure at the site of the wound, prevents blood loss it does not prevent blood flow through intact vessels. Therefore, unlike a tourniquet, a surgeon can perform an operation confident in the knowledge that tissue distal and proximal to the site of the surgery is receiving an adequate supply of blood. This means that there are no time limits on the length of the operation and that tissue surrounding or remote from the site of the operation will not be damaged.

In a further preferred embodiment of the invention said medical device is adapted to be disposable and is therefore, advantageously, prepared with, at least, the surface that makes contact with the patient sterilised. As will be apparent to those skilled in the art the inflatable aspect of the invention lends itself most suitably to being disposable. Indeed, the inflatable aspect of the invention lends itself to being provided in a foldable, and so pre-packed, form so that, when used, the disposable chamber can be removed from sterilised packaging and placed over the operative site or wound prior to surgery or treatment.

Advantageously, the inflatable medical device is ideally provided with a rigid or semi-rigid framework for positioning the inflatable material in spaced relationship with respect to the operative site, regardless of the pressure within the chamber.

In a preferred embodiment of the invention said chamber is provided with a pair of portals, one for each hand/arm of the surgeon. Advantageously, these portals are in the form of a glove/sleeve arrangement, which the surgeon can insert his hands into, and which sealingly engage with a part of the chamber from which they originate.

In any preferred embodiment of the invention a plurality of portals may be provided for enabling a plurality of instruments to be used in, or in association with, the inside of said chamber. For example, further portals may be provided to allow access for a catheter, laparoscopic device or the like.

During surgery the pressure inside said chamber is maintained at a level equal to or, more typically, greater than the tissue blood pressure at the site of operation or systolic pressure. Thus, typically, when the medical device is used the systolic pressure of the patient governs the level of pressure maintained within the chamber. Typically, throughout the operation the patient's systolic pressure is monitored in conventional fashion and appropriate adjustments are made to the pressure within the chamber. The systolic blood pressure of the patient will be continuously monitored by an intra-arterial cannula. This pressure reading will be fed to a computer which will regulate the gas compressor pump such that the chamber pressure is maintained, typically, but not exclusively, to just above systolic blood pressure.

Advantageously, the device of the invention provides an effective seal for use during surgery because the entire, or almost the entire, inner wall or skin of the chamber sealingly engages with the entire part of the body that is inserted into the chamber thus increasing the effective surface area of the seal. The device of the invention therefore not only provides sufficient pressure for a surgeon to be able to perform substantially bloodless surgery but it also provides sufficient pressure for the device to sealingly engage with a part of the body that has been inserted into the chamber.

In yet a further preferred embodiment of the invention the pressurised gas supply is oxygen or a mixture of oxygen and at least one other selected gas.

In yet a further preferred embodiment of the invention the medical device is for use on either humans or animals.

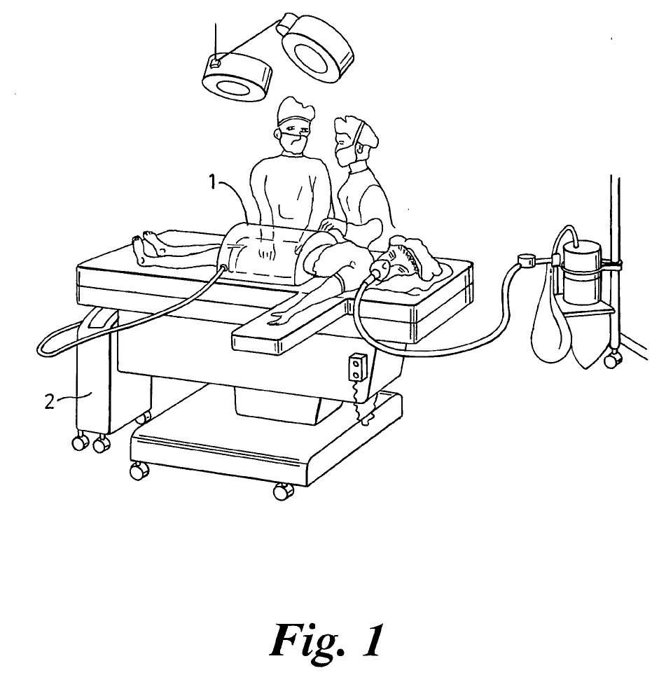

An embodiment of the invention will now be illustrated by way of example only with reference to FIGS. 1 to 4 wherein:

FIG. 1 shows an illustrative view of the medical device in accordance with the invention when used in a surgical environment;

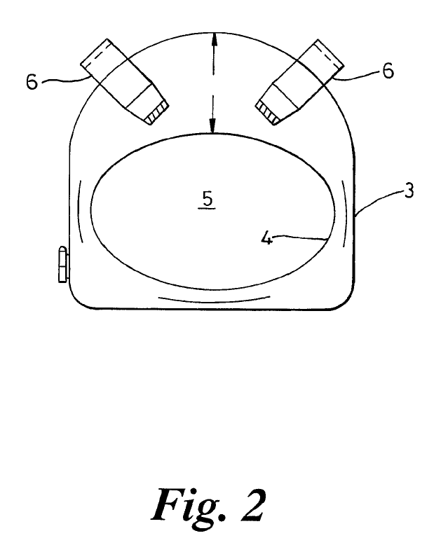

FIG. 2 shows a side end view of a medical device in accordance with the invention;

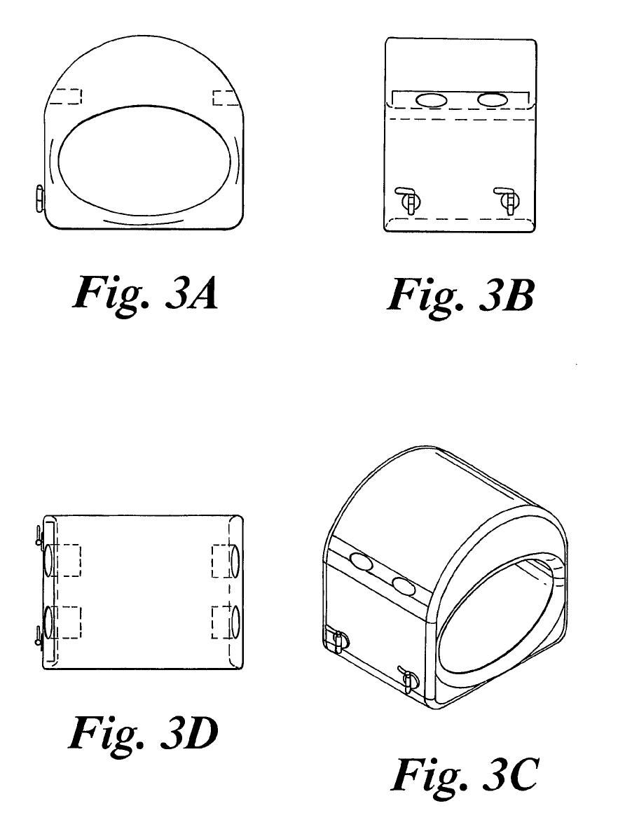

FIG. 3 shows various views of the medical device in accordance with the invention: FIG. 3A shows a side end view, FIG. 3B shows a lateral side view, FIG. 3C shows a perspective view, and FIG. 3D shows a plan view;

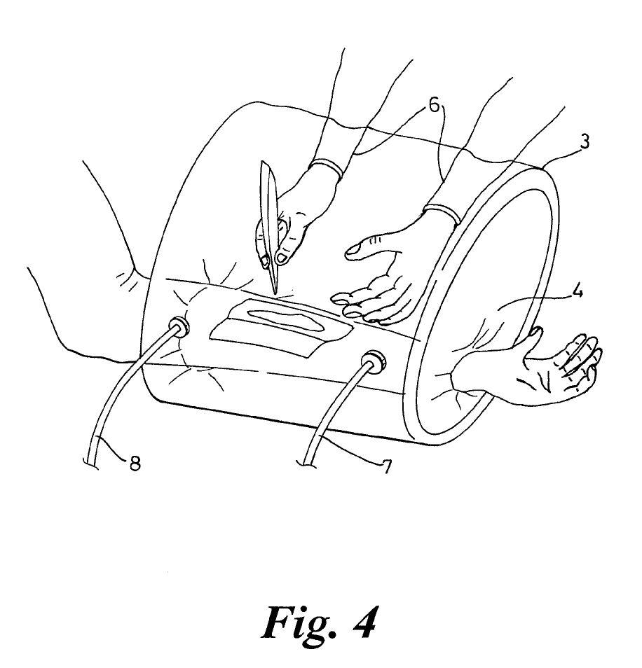

FIG. 4 shows a perspective view of a further medical device in accordance with the invention when used to perform limb surgery; and

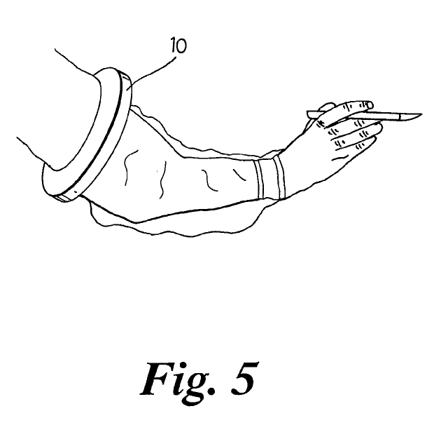

FIG. 5 shows a second embodiment of a second sleeve member according to the invention.

Referring to the figures and firstly to FIG. 1, there is shown a medical device when used in an operating theatre. The device 1 has been placed sealingly against the abdomen of the patient to be treated. Device 1 is in fluid communication with a pressurised gas supply 2 which is used to inflate device 1 and, subsequently, maintain the pressure inside device 1 at a substantially constant level. The remainder of the operating theatre is a standard and the patient is ventilated in the usual fashion using conventional equipment.

In FIG. 2 the medical device of the invention is shown in isolation but in an inflated condition. Thus, although not shown, the medical device 1 is in fluid communication with a pressurised gas supply.

Medical device 1 comprises a double skin arrangement including an outer skin 3 and an inner skin 4. The properties of these two skins vary as each is adapted for a specific purpose. Skin 3 and skin 4 may be made from the same material providing the material has the requisite properties. Alternatively, skin 3 may be made from a different material to that of skin 4 and, in any event, skin 3 is made from a material that has properties suitable for its intended purpose and skin 4 is made from a material that has properties suitable for its intended purpose. The device shown in FIG. 2 is typically for use during abdominal surgery and therefore comprises a suitably sized hollow core 5 through which the body of an individual is inserted. Once the medical device is inflated pressurised gas enters same and so exerts a pressure, indicated by the arrows, on the inner skin 4 and the outer skin 3. In this embodiment inner skin 4 is made from a different material to that of outer skin 3. Inner skin 4 is made of a thin and, typically, elastic material which, under pressure, clings to the skin of the patient to be treated. In contrast, outer skin 3 is made of a tougher material which, once inflated, resists any further deformation or stretching and so is able to help maintain the pressure within the chamber at a relatively constant level.

Although not shown in FIG. 2, pressure monitoring means are provided for monitoring the pressure within the chamber so that if the pressure should fall, as a result of leakage, more gas can be introduced into the chamber in order to maintain the pressure at a desired level. Moreover, venting means are typically associated with the chamber in order to facilitate the maintenance of this pressure. So, for example, should the pressure rise above a desired level then the venting means can be used in order to release some of the pressure within the chamber.

Also shown in FIG. 2 are portals 6. Typically, a pair of portals are provided on the left hand side of the chamber and an identical pair of portals are provided on the right hand side of the chamber so that the surgeon can choose on which side of the operating table he wishes to stand. Additionally, although not shown, further portals may be provided for introducing surgical equipment into the chamber for use during the surgical procedure. These latter portals comprise sealable entry points whereby a surgical instrument can be introduced to the inside of the chamber.

As those skilled in the art will appreciate, it may be desirable, immediately prior to introducing a piece of surgical equipment during an operation to allow the pressure within the chamber to rise slightly so that opening of a portal to introduce the equipment does not deleteriously affect the operation.

Referring to FIG. 3, the embodiment of the invention shown in FIGS. 1 and 2 is illustrated in greater detail with reference to different views. FIG. 3A is essentially the same view as shown in FIG. 2 except that the portals are shown on the upper sides of the chamber and penetrating into the chamber at right angles. FIG. 3B shows a side view of the chamber where it can be seen that a pair of spaced portals are provided at a desired height and below same there is provided a pair of venting means. FIG. 3C shows a perspective view of the chamber. The hollow core 5, through which an individual is inserted, is clearly seen, and further it can be seen that, once inflated, the cavity forms a dome-shaped structure above the body of the patient. FIG. 3D shows a plan view of the medical device and it can be seen that a pair of portals are provided on either side of the medical device.

Referring now to FIG. 4, there is shown a medical device adapted for use in limb surgery. This medical device essentially comprises a double-skinned chamber which is fashioned in the form of a sleeve. Once again, the inner skin 4 is adapted to sealingly engage with the body of the patient and so forms a tight, flattened seal against, in this illustration, a patient's arm. In contrast, the outer skin 3, once inflated, is spaced remote from inner skin 4 and so defines the pressurised chamber which, via gas supply means 7 and gas venting means 8, is maintained at a relatively constant pressure. Also shown in FIG. 4 are the sealed portals 6 through which a surgeon gains access to the pressurised chamber. In this illustration of the device a scalpel has been placed inside the chamber and the surgeon has used same in order to cut through inner skin 4 and also the tissue of the patient. Thus a single incision gives the surgeon access to a patient's tissue and, because of the properties of inner skin 4, the seal is not broken.

During use, a surgeon uses the pair of sleeved portals 7 in order to gain access to the inside of the device. The sleeved portals are shown in greater detail in FIG. 5 and in one embodiment of the invention the sleeves are provided as releasable attachments to the medical device which, in use, sealingly engage with the device in order to prevent pressure loss within the chamber.

In FIG. 5 the sleeve member comprises an elasticated cuff for positioning the sleeve about the hand/arm of the surgeon, it also comprises an annular sealing member 10 which sealingly engages with the rim defining the aperture of portal 7.

A range of portals can be built into the device of the invention to provide access for various devices such as, without limitation, a gas inlet with valve; a pressure monitoring gauge; arm portals; endoscopic or laparoscopic instruments; access for intravenous and fluid lines; access for anaesthetic endotracheal tube; in/out gas blocks; gas venting means; temperature and humidity monitoring means; and access for catheters.

Although not shown, in some embodiments, an inflatable structure is provided with skeletal supports in the form of rigid or semi-rigid hoops the purpose of which is to maintain the medical device in space relationship with the patient so ensuring that when the pressure within the medical device is reduced the device does not completely collapse and make contact with the patient.

Referring again to FIGS. 1 and 4, although not shown the device can be linked to a closed system gas generator which provides pressurised gas via a closed circuit system. Ideally, the closed circuit system is provided with a pressure monitoring means and associated valve means. Selective operation of valves in conjunction with the gas generator enables the pressure within the bag-like chamber to be maintained at a selected level. This closed system circuit provides for the efficient use of pressurised gas within the system.

Whilst the invention has been particularly described with reference to a medical device suitable for performing abdominal or limb surgery, it will be apparent to those skilled in the art that the shape and size of the medical device may take different forms according to the nature of the surgery that is to be performed.

The device is most advantageously used with a pressurised oxygen supply because oxygen promotes healing and therefore use of the device with this choice of gas not only enables bloodless surgery to be undertaken but also, at the same time, promotes healing of the tissue that has been operated upon.

In use, once a surgeon has performed surgery, he or she may, advantageously, lower the pressure within the chamber to a point at which blood leakage from vessels that have yet to be sealed or ligated is visible. In this way, the surgeon can see those vessels which require attention and then by increasing pressure within the chamber block any loss of blood from these vessels prior to taking steps to seal them. This procedure can be repeated however many times are necessary in order to successfully conclude the surgery.

If desirable, the device can be used to perform robotic surgery and, in this instance, a video camera will be provided within the pressurised chamber so that a surgeon, seated at a remote location, can view the operative field.

The device will be especially advantageous for the performance of Robotic Surgery:
(a) it will provide a clear operative field enhancing the visibility of vital organs. This in turn will render the operation more precise and reduce the damage to vital structures;
(b) it will make the performance of operative procedure faster as no time will be wasted in controlling the haemorrhage; and
(c) controlling haemorrhage during laparoscopic/endoscopic procedures manually or with robotic control requires removing the blood with some absorbent swab or a suction cannula. A dry bloodless field will obviate the need of an extra instrument to mop or suck the blood.

Further, we prefer to use oxygen to pressurise the medical device because, whilst unlikely, there is a theoretical possibility of air embolism if a large amount of air enters the venous circulation due to the insoluble nature of nitrogen. However, since we are using pure oxygen, which is highly soluble in blood, the chances of an embolism occurring are almost minimal. The patient will be monitored for features of embolism by transcutaneous oxygen saturation recording and monitoring of heart for presence of gas bubbles by a transesophageal echocardiography during the operation. Other potential benefits of using oxygen inside the pressurised chamber are:
(a) salutary effect of oxygen in promoting wound healing;
(b) enhancing collagen synthesis in the wound reducing the risk of wound disruption;
(c) prevention of wound infection; and
(d) improving viability of skin and muscle flaps in reconstructive surgery.

The invention therefore provides a medical device which enables the surgeon to perform bloodless or substantially bloodless surgery.

The said device may also find applications and benefits in the field of veterinary surgery.

1. A medical device for performing substantially bloodless surgery comprising an inflatable chamber having a through-bore adapted to receive a body part of an individual to be operated upon, which chamber in use, is in fluid communication with a pressurised gas supply for inflating same and maintaining pressure in said chamber at a selected level wherein, upon inflation of said chamber the inner and outer walls thereof are forced thereapart and the inner wall sealingly engages with the site to be operated upon and the outer wall defines at least a part of a pressurised chamber in which surgery can take place; and further wherein said chamber includes at least one portal that allows access to the inside of the chamber, without significantly affecting the maintenance of said pressure, whereby said surgery can take place.

2. A medical device according to claim 1 wherein the inner wall of said chamber is made from a material that can be cut with a scalpel.

3. A medical device according to claim 1 wherein the outer wall is made of a material that, upon reaching a certain size, will resist further deformation.

4. A medical device according to claim 1 wherein the inner wall is made from a different material to the outer wall.

5. A medical device according to claim 1 wherein said device is provided with a pressure monitoring means in order to monitor the maintenance of said pressure at said selected level.

6. A medical device according to claim 1 wherein said device is provided with venting means for venting said pressurised gas from said chamber.

7. A medical device according to claim 1 wherein said device is provided with a closed circuit gas system whereby pressurised gas delivered to said chamber is returned to said pressurised gas supply before being reused to maintain the pressure inside said chamber.

8. A medical device according to claim 1 wherein said device is adapted to be disposable.

9. A medical device according to claim 1 wherein at least the inside of said chamber, that is the part that makes contact with the patient, is provided in a sterilised form.

10. A medical device according to claim 1 wherein said device is provided with a rigid or semi-rigid framework for positioning the inflatable device in a spaced relationship from the site to be operated upon, regardless of the pressure within the chamber.

11. A medical device according to claim 1 wherein said device is provided with a pair of portals.

12. A medical device according to claim 1 wherein said portals are in the form of a glove/sleeve arrangement which sealingly engages with a part of the chamber from which it/they originate.

13. A medical device according to claim 1 wherein said device comprises a plurality of portals for enabling a plurality of instruments to be used in, or in association with, the inside of said chamber.

14. A medical device according to claim 1 wherein said chamber is, at least in part, transparent so that a surgeon can see into the chamber in order to perform surgery.

15. A medical device according to claim 1 wherein the entire device is transparent.

16. A medical device according to claim 1 wherein the device is provided in a prepacked form.

17. A medical device for performing substantially bloodless surgery comprising an inflatable double-skinned chamber which, in use, is in fluid communication, via a single inlet means, with a pressurised gas supply for inflating same and maintaining pressure in said chamber at a selected level; said chamber being adapted to be placed sealingly against or about an individual to be treated and including at least one portal that allows access to the inside of said chamber, without significantly affecting the maintenance of said pressure, whereby said individual can be treated characterised in that;
said chamber comprises an inner and an outer skin, the inner skin being made of a relatively thin material that can be cut with a scalpel and the outer skin being made of a material that resists deformation on inflation whereby on inflation of said chamber said inner and outer skins are forced thereapart whereby said inner skin sealingly engages with the site to be operated upon and the outer skin defines a pressurised cavity in which surgery takes place.
